(19) United States
(12) Patent Application Publication
(10) Pub. No.: US 2004/0261793 A1
(43) Pub. Date: Dec. 30, 2004

(54) Lung-ventilator adapted for patients under intravenous anaesthesia

(76) Inventors: Stefan Stromberg, Sigtuna (SE); Nils Olof Eriksson, Enskede (SE)

Correspondence Address: BANNER & WITCOFF, 1001 G STREET N W, SUITE 1100, WASHINGTON, DC, 20001, US

(21) Appl. No.: 10/481,460

(22) PCT Filed: Jun. 17, 2002
(86) PCT No.: PCT/SE02/01169

(30) Foreign Application Priority Data
Jun. 19, 2001 (SE) 0102173-2

(51) Int. Cl. A61M 16/00; A62B 7/00

(52) U.S. Cl. 128/204.18; 128/204.23; 128/205.24

(57) ABSTRACT

The invention relates to a lung ventilator that includes a control unit (3a), at least one gas delivery hose (4), and a patient-allocated or -related device, wherein one end part of the hose faces towards the control unit and the other end part of the hose faces towards said device. The control unit (3a) includes means for passing an inhalation gas flow or an insufflation gas flow to the patient, and an expiration valve (6) is adapted to allow expiration gas to flow to the free surroundings, said expiration valve being controllable to either one of two functional states. A reserve volume (7′) in a container (7) or bag is adapted to generate manually a gas flow, intended to an insufflation phase, wherein this gas flow can be actuated by applying pressure to the container temporarily and over or during a short tie period. The thus pressurised reserve volume in the container or bag is delivered to the hose (4c), which is connected directly to the patient-allocated or related device (5, 6).

### FIELD OF INVENTION

The present invention relates generally to a lung ventilator and more specifically to a lung ventilator of the kind, which includes at least one control unit, at least one gas delivery line or hose, and a patient-allocated or -related device.

The lung ventilator is particularly adapted for use with such patients that are treated under intravenous anaesthesia.

The control unit is normally adapted to achieve “mechanical” lung ventilation, principally based on the ability to control the supply of insufflation gas to a patient during selected periods or cycles, associated with or related to a normal breathing rhythms.

Gas delivery lines, normally consisting of comparatively long hose sections, are connected to various short channels. However, for the sake of simplicity, each of these lines will be referred to as a “gas delivery hose” in the following descriptive portion of this application.

More particularly, the present invention is intended to find application in lung ventilators of the kind that can be switched between at least two different functional modes.

A first functional mode is designated “Spontaneous/Manual” mode, and a second functional mode is designated “Controlled Mechanical Ventilation” mode, abbreviated as (CMV).

In respect of an application, described in more detail hereinafter, the term gas delivery hose is used in reference to a proposed application and also in reference to a proposed embodiment, beneficially with the use of a hose for delivering inhalation gas or insufflation gas, or air, or a mixture of air with some other gas, such as oxygen gas. The term hose also refers to a hose intended for controlling an expiration valve to either one of two states. The term hose is also used to refer to one or more further hoses, intended for measuring different criteria.

In the case of the lung-ventilating arrangement, according to the present invention, one end portions of respective hoses face towards the control unit or the ventilator, while the other end portions of said hoses face towards the patient-allocated or -related device and co-act with the ventilator and said device respectively in a known manner.

Moreover, one of these gas delivery hoses, hereinafter referred to as a first hose, shall enable a spontaneous inhalation gas flow to be passed or led to the patient, or to drive from the ventilator a gas flow intended for mechanical insufflation.

The arrangement also enables the use of a valve, which is located in the close proximity or adjacent of the patient-allocated or -related device and which is adapted to allow expiration gas to pass out to the free surroundings in a controlled fashion.

The expiration valve can be controlled to adapt one of its two functional states from the control unit. This control may conveniently be achieved by pressurising a second of said gas delivery hoses, hereinafter referred to as a second hose.

The inventive lung ventilating arrangement is expected to find particularly suitable application with patients under treatment and to whom a pain-relieving substance or some similar substance has been administered intravenously.

More particularly, the invention is intended to be able to afford advantages during anaesthetising and recovery phases, during which there may be reason to support spontaneous breathing in the selected functional mode “Spontaneous/Manual” temporarily by manually initiating and effecting an insufflation phase.

A system, in which the present invention is intended to find a particular convenient application, is known to the art and described in the Swedish Patent Application Serial Number 99 02051-3 (corresponding to International Patent Application PCT/SE 00/01067, with Publication No. WO-A1-00/74757).

### DESCRIPTION OF THE BACKGROUND ART

A large number of different designs of lung-ventilating arrangements of the aforesaid kind, that normally include a lung-ventilator unit with an associated control unit, are known to the art.

Control units of the kind meant here are of relatively complex design, so as to be able to sense/detect and control different patient-related criteria, and it is not unusual to “customise” the required functions in the lung-ventilator unit with respect to chosen fields of application and to chosen other required conditions.

Lung ventilator units with associated control units can normally be set to one of a number of available functional modes.

The present invention is intended to find its application when the lung-ventilator unit is set to a functional mode designated “Spontaneous/Manual” mode.

This functional mode is based on the ability of the patient to breathe spontaneously with periodic inhalation and expiration phases, and on the assumption that only occasionally the well-being of the patient may require the application of additional measures, namely the delivery to the patient of a manually initiated insufflation phase.

To this end, there is provided a flexible container or bag, which is filled with gas or air, wherewith air is delivered manually via the insufflation phase, by manually compressing the container or bag so as to press from the container or bag all or part of a reserve gas volume enclosed therein.

Lung-ventilating arrangements of the kind intended in this document or application also provide another functional mode, designated “CMV” mode, in which the patient is administered successively with insufflation phases mechanically and via the control unit.

An earlier known arrangement, constructed in accordance with the aforesaid, can be used for a general lung-ventilatory purpose on patients who breathe spontaneously, via a first functional mode, on the one hand, and for the use on patients who are in an anaesthetic state or a state of deep anaesthesia via a second functional mode, on the other hand.

In known arrangements that include a lung-ventilating unit or lung ventilator there are used a number of gas delivery hoses, at least two, and a patient-allocated or -related device that includes a valve.

In this respect, there is used a first gas delivery hose, which extends between the lung ventilator and the patient, and which is adapted for delivering gas or air for spontaneous inhalation, or to deliver insufflation gas or an insufflation gas mixture to the patient mechanically and/or manually, with the first hose coupled to a patient-allocated and patient-proximate connecting device, that includes an expiration valve connected to the lung ventilator.

Reference is here made to the contents of the Swedish Patent Application Serial No. 99 02051-3 in this regard.

Different kinds of patient-connecting devices exist, such as facemasks, tracheal tubes and larynx masks, for instance.

It is also known, within the field concerned, the use of lung-ventilating units on living creatures in a non-anaesthetised state, such as from Patent Publication US-A-3,961,627, to include, in the proximity of a creature or a patient in the direction of flow of the gas, a control valve and a measuring device that includes a necessary sensor unit, in the form of a device that determines gas flow and gas pressure.

The valve drive mechanism may, for instance, comprise a servomotor, that receives control signals from a valve-controlling unit.

It is also known to provide a control unit, which, similar to devices required for evaluating therapeutic and/or diagnostic criteria, of the nature described and illustrated more specifically in the Swedish Patent Application Serial No. 99 01688-3 (corresponding to International Patent Application No. PCT/SE 00/00910, with Publication No. WO-A1-00/67820.)

It is also known to allow the insufflation gas either to consist solely of a pure air or an air mixed to a greater or lesser extent with oxygen gas, or solely oxygen gas and/or additives thereto. Consequently, mention is made in the following description to a number of hoses, which are described as gas delivering hoses, regardless of whether the hose concerned conducts a gas flow or whether said hose functions solely to pressurise an expiration valve, in order to bring the valve to a closed state.

A further example of the known technology is shown in FIG. 1.

This figure shows that a lung ventilator is able to generate a pulsating control flow during the functional mode “CMV”, wherein the control flow passes a valve unit and shunts or deflects a part to an elastic container or bag that serves to provide a reserve volume, and wherein the pulsating flow acts against a rigid container.

Arranged within the rigid container is an elastic bellows-like device, which is compressed and expanded by a pulsating flow.

Enclosed in the bellows-like device is a volume of air, intended for inhalation or insufflation, said air volume is connecting with a patient-associated pneumatic system, to which a chosen gas volume is delivered per unit of time.

More particularly, it is evident from the known technology that the ventilator “V” is coupled during the functional mode “CMV” to create a pneumatic drive system, which actuates correspondingly a patient-allocated or -related pneumatic system via an interface.

In this case, the interface consists of a unit designated “Bag in Bottle”.

### SUMMARY OF THE PRESENT INVENTION

Technical Problems

When taking into consideration the technical deliberations that a person skilled in this particular art must make in order to provide a solution to one or more technical problems that he/she encounters, it will be seen that it is necessary initially to realise the measures and/or the sequence of measures that must be undertaken to this end on the one hand, and, on the other hand, to realise which means is/are required in solving one or more of said problems. On this basis, it will be evident that the technical problems listed below are highly relevant to the development of the present invention.

When considering the present state of the art, as described above with regard to a lung ventilator that includes a control unit, at least one gas delivery hose and a patient-allocated or -related device, wherein one end portion of said hose faces towards the control unit while the other end portion of said hose faces towards the patient-allocated or -related device, and where the control unit includes means for delivering to the patient an inhalation or an insufflation gas flow, and where there is included an expiration valve that is adapted to allow expiration gas to flow freely to the surroundings, and where the expiration valve can be controlled to one of two functional states, it will be seen that a technical problem resides in the ability to significantly simplify the construction of the lung ventilator.

It would seem that a technical problem also exists in providing structural elements that are able to eliminate the presence of a drive system on the one hand, and a slave system, in the form of a patient-related pneumatic system, on the other hand.

Another technical problem would seem to reside in the ability to provide constructional descriptions that enable a rigid container with its enclosed bag (Bag in Bottle), used in earlier techniques, to be eliminated without any deleterious affect on the function of the lung ventilator.

Another technical problem resides in the ability to provide constructional details, which enable the pneumatic drive system to operate as said patient-allocated pneumatic system, with the aid of a valve arrangement, that includes a plurality of valve functions.

In addition, it will be seen that a technical problem resides in the ability to create conditions, in which a valve arrangement enables a number of valve functions to be actuated in the lung ventilator, by which it has been possible to connect these valve functions directly to a patient-allocated or -related device and to actuate said device.

Another technical problem resides in the ability to realise the significance of and the advantages that are afforded when an air flow, generated by the lung ventilator, can be utilised effectively, and to utilise said air flow to replenish a reserve volume enclosed in an elastic container or bag, in addition to providing a manual insufflation phase.

Another technical problem resides in the ability to realise the significance of and the advantages associated with using the content of a flexible container or bag as a requisite disposable reserve volume, intended for a manually initiated insufflation phase, and therewith allow said container or bag to be connected to a valve arrangement.

Another technical problem resides in the ability to realise the significance of and the advantages associated with the creation of conditions that enable said container reserve volume to be replenished at every requirement to an extent such as to generate a gas flow intended for a manual insufflation phase, through the medium of a valve arrangement that includes a number of valve functions. The gas flow for the manual insufflation phase can be actuated by exerting pressure on the container temporarily and over a short time duration, wherewith the reserve volume, held by the container or bag, is delivered, under the pressure generated, to said hose, that is connected directly to the patient-allocated or -related device, via said valve arrangement.

Another technical problem resides in the ability to realise the significance of and the advantages afforded by connecting the container to a T-coupling belonging to an insulation hose, with an associated valve arrangement.

Another technical problem resides in the ability to realise the significance of and the advantages that are afforded by coupling the control unit to a valve arrangement that has a number of valve functions, or including such a valve arrangement in the control unit, wherein said valve arrangement functions, through the medium of the container or bag overpressure and through gas flow, a cylinder-housed piston to cause, among other things, a gas flow, serving as a manual insufflation phase, to be delivered to said patient.

Another technical problem resides in the ability to realise the significance of and the advantages that are afforded by allowing the valve arrangement to take a first functional setting, in which gas can flow to the container or bag and also from the ventilator.

Another technical problem resides in the ability to realise the significance of and the advantages afforded by providing said valve arrangement with a number of selected and co-ordinated valve functions, of which one will be able to actuate a patient-allocated or -related and patient-proximal expiration valve.

Another technical problem resides in the ability to realise the significance of and the advantages that are afforded by allocating to the control unit a valve, which is adapted and set so that the gas flow required for the health of the patient and the gas flow required to replenish the container or bag can be delivered to the hose and to the patient-allocated or -related device respectively also in the functional mode “Spontaneous/Manual”.

Still another technical problem resides in the ability to realise the significance of and the advantages that are afforded when a chosen resistance in the pneumatic system, such as the influence of the opening resistance of a one-way valve, is adapted to control the container or bag replenishing rate.

Yet another technical problem resides in the ability to realise the significance of and the advantages afforded by allowing a chosen continuous flow through the one-way valve to be dimensioned, while taking into account and while well-balancing the resistance situation within the pneumatic system, and, in addition, to allow the valve arrangement to be actuated by the influence of a flow from the container or bag.

Solution

The present invention takes as its starting point a lung ventilator that includes a control unit, at least one gas delivery hose, and a patient-allocated or -related device, wherein one end part of the hose faces towards the control unit and the other end part of the hose faces towards said device, wherein the control unit includes means for delivering to the patient a gas flow intended for inhalation or insufflation, wherein an expiration valve is adapted to allow expiration gas to flow to the free surroundings, and wherein the expiration valve can be controlled to take one of two functional states.

With the intention of solving one or more of the aforesaid technical problems, it is proposed, in accordance with the present invention, that a pneumatic system actuated by the control system is coupled, together with a patient-allocated or -related pneumatic system, via a valve arrangement, and that a flexible container or bag, intended to hold a reserve gas volume, is coupled to the valve arrangement so as to provide a manually initiated insufflation phase, through the medium of said valve arrangement.

In accordance with one embodiment, it is proposed that the expiration valve shall be controlled via an overpressure in a hose and orientated close to or adjacent to the patient.

It is also proposed that a reserve volume in the container or bag shall be adapted to generate a gas flow, which is intended for a manual insufflation phase, and which can be activated by pressurising the container or bag temporarily and for a short duration of time, and that the container or bag included reserve volume is delivered to the hose via said valve arrangement as the container or bag is subjected to pressure.

By way of proposed embodiments, that lie within the scope of the inventive concept, it is also proposed that the container or bag is connected to a T-coupling, including the valve arrangement, and belonging to said insufflation hose.

It is also proposed that the control unit, including the valve arrangement, controls and allows the passage of insufflation gas to the patient.

In accordance with the invention, when the valve arrangement is set to a valve function in one state, a limited flow of gas is able to pass to the container or bag for replenishment thereof.

In particular, the valve arrangement shall include a number of coordinated valve functions that can be actuated by the movement of a piston.

A valve, allocated to the control unit, shall be adapted to deliver an insufflation flow to the hose and to the patient-allocated or -related device.

It is also proposed that a proportion of said insufflation flow shall be delivered to the container or bag via said valve arrangement and a selected valve function.

There is also proposed the use of a valve arrangement that includes a number of valve functions, which can be selected through the medium of the movement of a piston in a cylinder, wherein the piston movement is controlled by a gas flow in the direction of movement of said piston.

It is also proposed that, primarily, a chosen opening resistance of a one-way valve shall be adapted for controlling the rate at which the container or bag is replenished with gas.

It is also proposed that a chosen continuous flow for the one-way valve to-gether with a well-balanced pneumatic resistance relation in the pneumatic system shall mutually be adapted so as to provide selective pressurisation of the container or bag content.

Advantages

Those advantages primarily associated with an inventive lung ventilator reside in the creation of conditions in which a volume of gas, intended for an insufflation phase, can be stored in a flexible container or bag, with the aid of a valve arrangement and related valve functions, wherein solely the flexible container or bag will enclose a reserve volume required for a manual insufflation phase.

More specifically, the invention is concerned in the elimination of a “Bag in Bottle” unit, by allowing the ventilator to deliver a constant flow of insufflation gas in the functional mode “Spontaneous/Manual”.

The primary characteristic features of an inventive lung ventilator are set forth in the characterising clause of the accompanying claim 1.

### BRIEF DESCRIPTION OF THE DRAWINGS

An earlier known lung ventilator will now be described in more detail with reference to a pertinent figure, and a lung ventilator, according to the invention, will be described in more detail with reference to the accompanying drawings, in which;

FIG. 1 is a principle illustration of an earlier known lung ventilator together with coupled devices;

FIG. 2 illustrates the lung ventilator coupled in accordance with the present invention;

FIG. 3 is a schematic illustration of a lung ventilator supplemented in accordance with the present invention;

FIG. 4 is a principle diagram of a lung ventilating unit and/or control unit, which includes a number of invention related and requisite functions so as to be able to provide the significant characteristic features of the present invention;

FIG. 5 illustrates a valve arrangement actuated by a control unit, in a state in which the reserve volume of a container or bag is emptied by an overpressure acting upon said container or bag, to generate manually an insufflation gas phase;

FIG. 6 shows the valve arrangement in a state in which an empty container or bag can be supplied successively with a maximised reserve volume, via valve functions in the valve arrangement;

FIG. 7 illustrates an example of means for setting the valve arrangement into either one of two different function-module-related states; and

FIG. 8 is a part view of the valve arrangement intended to illustrate a chosen design of a seal between a cylinder-housed piston, which is moved up and down by a gas flow, and a surface part of said cylinder.

### DESCRIPTION OF EMBODIMENTS AT PRESENT PREFERRED

It is pointed initially that we have chosen to use in the following description of embodiments at present preferred and including significant characteristic features of the invention and illustrated in the figures of the accompanying drawings special terms and terminology, with the intention of illustrating the inventive concept more clearly.

However, it will be noted that the expressions chosen here shall not be seen as limited solely to the chosen terms used in the description, but that each chosen term shall be interpreted as also including all technical equivalents that function in the same or at least essentially the same way so as to achieve the same or essentially the same intention and/or technical effect.

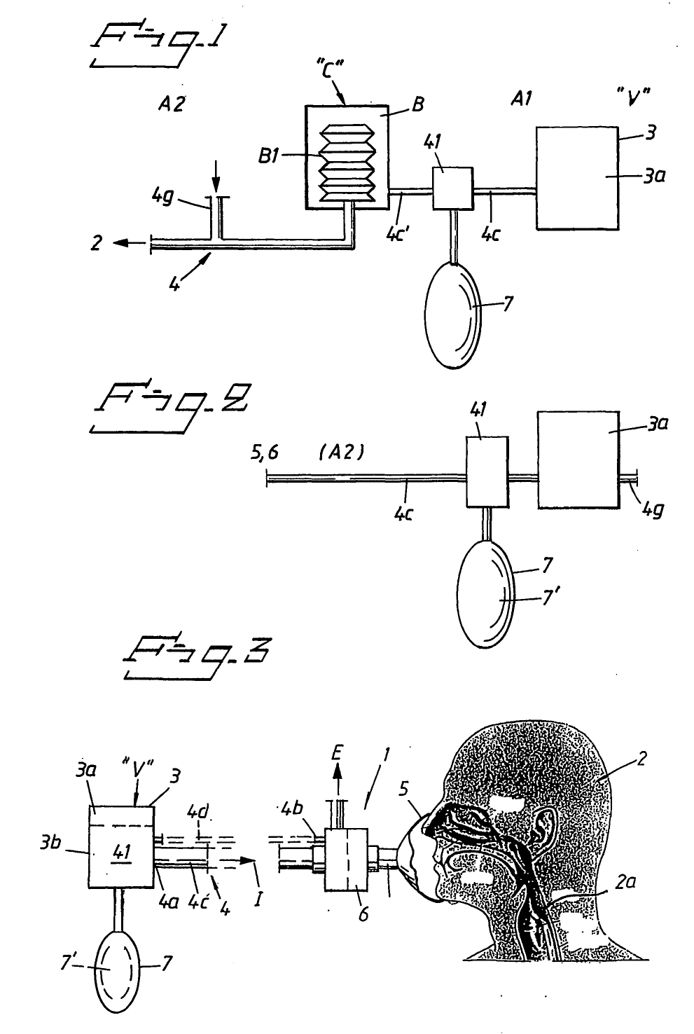

Shown schematically in FIG. 1 is an earlier known lung ventilator “V”, that includes a lung-ventilating unit 3 and a control unit 3a and at least one hose 4c, that extends from the lung ventilator to a valve arrangement 41, which includes or is configured as a T-coupling for connection to a flexible container 7 and bag and to control an outer pressure in a container “B” via an extension 4c′ of the hose or line 4c, said container “B” including a bellow-like device B1, connected to a system intended for inhalation or insufflation.

As illustrated in FIG. 1, the known principle is based on two coordinated pneumatic systems, a system “A1”, created by the lung ventilator or control unit, and a patient-related system “A2”.

The system “A1”, related to the lung ventilator, is adapted to actuate the patient-related system “A2”.

The system “A1” is driven, via the control unit 3a, and delivers air to and removes air from the rigid container “B” and thereby compress or expand the bellows-like arrangement “B1” correspondingly.

The bellow-like arrangement “B1” is included in the patient-related system “A2” and creates the air flow required for breathing purposes.

Air or gas is delivered to a person 2 from the bellow-like device “B1” through a hose 4, and a pulsating gas quantity, a gas volume is accommodated in the bellows-like arrangement “B1”, such that insufflation phases can be delivered consecutively to the patient 2.

When the ventilator “V” or the control unit 3a is set to a “CMV” functional mode the control unit is driven in the aforedescribed manner.

Gas is delivered to the patient via a line section 4g and required pulsation is effected via a unit “C”.

The ventilator “V” is fully disconnected in the functional mode “Spontaneous/Manual” and the patient breathes spontaneously totally via the pneumatic system “A2”.

In the case of the present invention, illustrated in FIG. 2, a pneumatic system actuated by the control unit 3a is coupled to the patient-related pneumatic system (A2) via a valve arrangement 41, wherein a container 7 or bag, that accommodates a reserve volume 7′, is coupled to the valve arrangement 41, so that a manual insufflation phase can be provided via said arrangement.

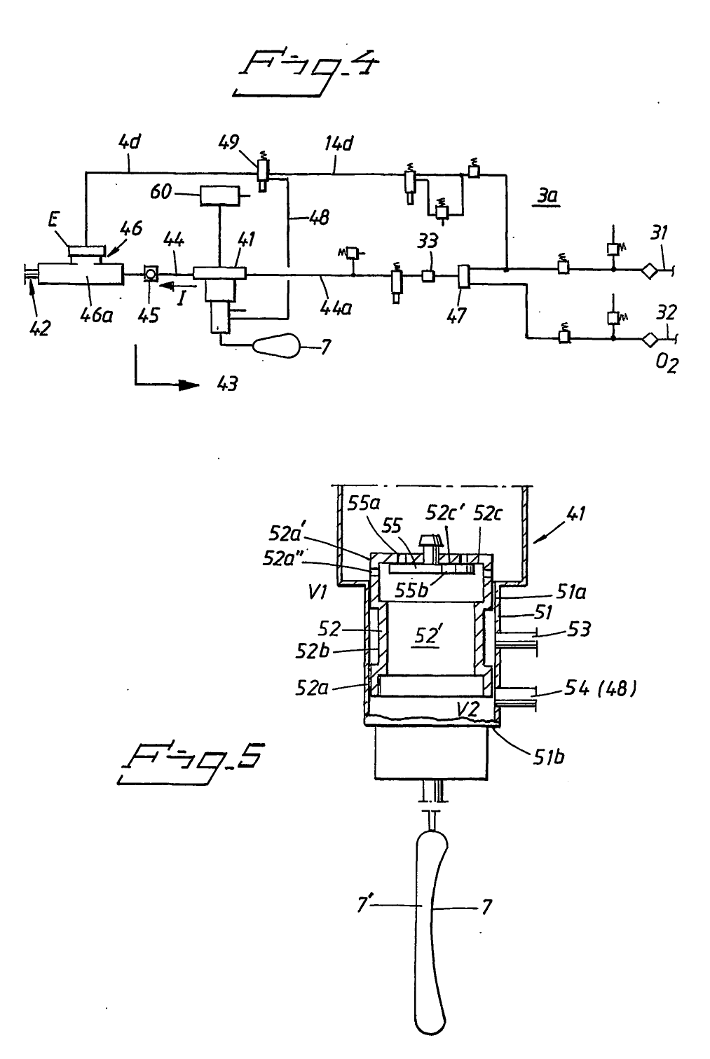

FIG. 3 illustrates the lung-ventilator unit 3, which includes a control unit 3a, at least one gas delivery hose 4c, and a patient-allocated or -related device 5, wherein one end part 4a of the hose faces towards the control unit while the other end part 4b of said hose faces towards the patient-allocated or -related device.

The control unit 3a may conveniently include means for delivering to the patient 2 an inhalation or insufflation gas flow, wherein an expiration valve 6 is adapted to allow the expiration gas to flow to the free surroundings, and wherein said expiration valve can be controlled to take one of two functional settings.

The inventive apparatus includes said container 7 or bag, which accommodates said reserve volume 7′ intended for generating a gas flow through the valve arrangement 41 to provide an insufflation phase as required, wherein it shall be possible to actuate an insufflation phase by exerting pressure on the container 7 or bag by a manual compression, temporarily and for or during a short time period.

The thus pressurised reserve volume 7′, in the container 7 or bag, is delivered to the hose 4c, which is connected directly to the patient-allocated or -related device 5.

The illustrated container 7 or bag is connected to a T-coupling belonging to the insufflation hose and included in said valve arrangement 41.

The control unit 3a may include said valve arrangement 41, which allows insufflation gas to pass to the patient in response to the overpressure generated in the container or bag.

The valve arrangement 41 is adapted to allow gas to flow to the container 7 or bag in a first state or position of the valve arrangement.

The valve arrangement 41 includes a number of valve functions.

Primarily, a selected opening resistance of a one-way valve shall be adapted for controlling the rate at which the container or bag content is replenished.

A selected continuous flow through the one-way valve and a well-balanced pneumatic resistance relation in the pneumatic system are, together, adapted to limit pressurisation in the container or bag.

Connected to the device 5, or facemask, is a valve arrangement 6, the nature of which will be described in more detail hereinafter and which is described and illustrated more comprehensively in a Swedish Patent Application Serial No. 00 04066-7, the contents of this application being intended to constitute part of the present application and also part of the present description.

One end-parts 4 of the hoses 4 face towards the control unit, while the other end parts 4b of said hoses face towards the patient-allocated or -related device, or more precisely towards an expiration valve 6.

The control unit 3a includes means for periodically delivering consecutive insufflation phases in the functional mode “CMV” via one of the gas delivery hoses 4c, where a valve arrangement, in the form of an expiration valve 6 situated proximal to the patient-allocated or -related device 5, is adapted to allow expiration gas to flow periodically to the free surroundings.

The valve arrangement 6 can be caused to take one of two available settings by the control unit 3a, by virtue of pressurising a second of said gas delivery hoses, namely the hose referenced 4d.

The invention aims at readily providing a brief manually initiated insufflation phase under otherwise good spontaneous breathing conditions.

Also shown in FIG. 3 is said container 7 or bag, which includes said reserve gas volume 7′ for generating a gas flow, intended for a temporary insufflation phase, wherein the flexible container 7, comprising an elastic or flexible rubber bladder, is instrumental in providing the possibility of placing the hose 4c under pressure, temporarily and for a short duration of time, for the passage of gas while increasing the pressure in the hose 4d.

The gas flow, intended for the insufflation phase, is referenced “I” and the gas flow, intended for the expiration phase, is referenced “E”.

A proposed embodiment of said valve arrangement 41, having a proposed coupling in the control unit 3a, will now be described in more detail with reference to FIGS. 4 to 8 inclusive.

The coupling is apparent from the diagrammatic illustration of FIG. 4.

This illustration shows the use of an expiration valve 46, which is incorporated in a casing 46a, connected to the patient 42 and to a first gas delivery hose, designated insufflation hose 44.

The expiration valve 46 can be caused to take one of two fixed states via a line or hose 4d intended for gas pressure.

This line or hose 4d is placed under pressure via a first function-mode-related valve unit 49, for closing the expiration valve 46 periodically in the functional mode “CMV”, by periodically placing a line 4d under pressure.

In this case, the valve unit 49 takes a state corresponding to the functional mode “CMV” and therewith shuts out from the valve arrangement 41 any pressure variation that may possibly occur on a line 48.

The valve 49 can also take a “Spontaneous/Manual” mode state, wherein only pressure variations, that occur on line 48, are delivered to line 4d and the expiration valve 6.

When required, a gas delivery hose 44, includes a check valve 45, is used so that an inhalation gas can be delivered to the patient 42, and, when necessary, also insufflation gas during the manual insufflation phase, from the control unit 3a and a valve arrangement 41, included in said unit 3a, therewith creating an adapted opening resistance, should the pneumatic system so require, in order to achieve the inventive function.

This check valve 45 may conveniently be incorporated in the valve arrangement 41, although this conceivable embodiment has not been shown in detail.

The hose 44 is connected to the valve arrangement 41 in a known manner, and the arrangement 41 is connected to an air supply 31 and to an oxygengas supply 32 via a line 44a.

The necessary mixing of these gases is effected in a mixer valve 47.

In the “CMV” functional mode, insufflation gas is delivered periodically for mechanical ventilation, whereas in the functional mode “Spontaneous/Manual” a continuous delivery of insufflation gas takes place.

More specifically, the invention teaches the use of a needle valve 33, intended for regulating the flow of gas from the lung ventilator “V” (43) during the functional mode “CMV” in a known manner, but above all also during the functional mode “Spontaneous/Manual”, in accordance with the proposals made, in which a purely constant gas flow is intended.

In this regard, the gas flow shall be adapted to at least satisfy the need of spontaneous breathing in respect of the patient 2, and to replenish the container 7 or bag within a chosen time section. One requirement is to allow the adapted gas flow to be delivered in an excess quantity.

This excess quantity, or surplus, would pass through the expiration valve 6.

This setting serves the purpose of replenishing the container 7 or bag against the effect of a pneumatic resistance prevailing in the system, such as the patient's resistance, the resistance of the expiration valve, and/or the effect of the opening resistance of the check valve 45.

The regulated continuous flow, through the needle valve 33, and a well-defined resistance relationship in the system enables the container 7 or bag to be replenished and placed under pressure in a selected and adapted fashion.

The construction of the proposed valve arrangement 41 will now be described in more detail with reference to FIGS. 5 to 8 inclusive.

The valve arrangement 41 includes a cylinder 51 and a piston 52, the latter can move freely up and down in the cylinder.

An effective seal is necessary between the piston 52 and an inner cylindrical surface 51a of said cylinder.

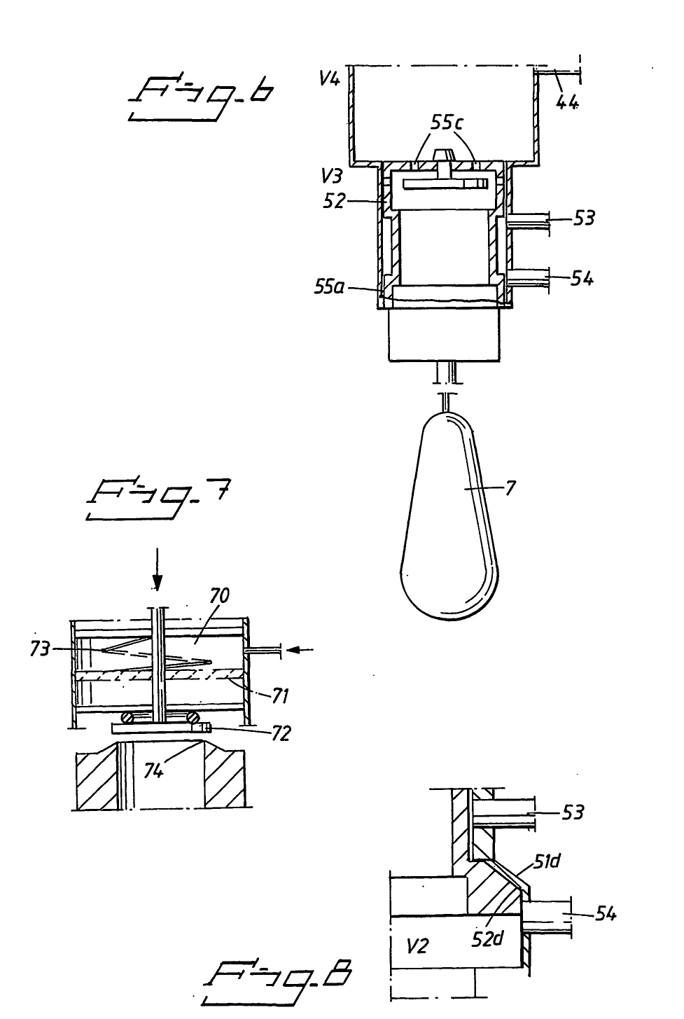

These are not shown in FIGS. 5 to 6 for the sake of clarity, although FIG. 8 shows requisite oblique sealing surfaces in the form of a surface part of the cylinder and a surface part of the piston, as described in more detail below.

The cylinder surface 51a opens, via two channels 53, 54, to an upper channel, which is used as an air venting channel 53, and a lower channel, which is used for transferring 54 (48) to the expiration valve 6 an overpressure generated in the container 7 or bag.

The channel 54 is thus connected directly to the line 48.

The piston 52 is able to move vertically up and down in the cylinder 51. It will drop down onto an abutment 51b or said seal under its own weight, among other things, and take a lower position, wherein the piston is lifted up to an upper position by a gas flow acting from beneath.

The lower position of the piston, according to FIG. 6, is taken during the functional mode “CMV” and also during that part of the “Spontaneous/manual” functional mode, in which no manual insufflation is applied.

The upper position, shown in FIG. 5, is taken through the medium of a gas flow from the container during the manual insufflation phase, so as to conform to the function of the invention.

The piston 52 has a general thimble-like shape and includes an outer cylindrical surface 52a that includes a peripheral opening 52b, which in one position of the piston, a lower position according to FIG. 6, connects an overpressure channel 54 (48) with the air venting channel 53.

This arrangement ensures that a cavity 6d, in the expiration valve 6. will be evacuated when the piston 52 is in its lower position, and that no overpressure, capable of closing the valve 6, acts on the surface 6c.

In the upper position of the piston 52, shown in FIG. 5, the overpressure channel 54 is connected to the line 58 for transferring the pressure generated by the container 7 or bag to the line 4d and the expiration valve 6, which is then held closed, via a first function-mode-related valve 49.

This takes place for as long as gas flows from the container 7 or bag, through the piston 55 and into the hose 44 and in a direction towards the patient 42.

The upper part of the piston, or the piston top 52c, co-acts with a one-way valve 55.

A cylindrical part 52a′, close to the piston top, includes holes or openings 52a″ so that, with the piston 52 in its upper position, the reserve volume 7′ is able to pass to the hose 44 as a manually activated insufflation phase.

FIG. 5 shows the valve arrangement 41 belonging to the control unit in a state in which the reserve volume 7′ in the container 7 or bag is emptied manually, either completely or partially, via an overpressure generated in the container or bag by hand.

The lower top surface 52c′ of the piston or piston unit 52 includes a flat one-way valve element 55, that includes a disc-shaped valve body 55b situated in the cavity 52′ of the piston unit 52.

The gas flowing from the container 7 or bag can cause the valve body 55b to move to an upper position, in which the holes 55c (see FIG. 6) are sealed off, and is able to move under its own weight to a lower position, in which the holes 55c are exposed.

The sealing position (FIG. 5) is taken automatically and initially in response to overpressure generated by the container 7 or bag, said overpressure and gas flow also lifting the piston unit 52 to an upper position and therewith enabling a manually initiated insufflation gas flow to pass from the container 7 or bag to the patient 42, via the valve arrangement 41 and the hose 44.

FIG. 6 shows the valve arrangement 41 with the piston unit 52 in a downwardly displaced position and in a freely lying state, in which the valve body 55b of the valve device 55 is downwardly displaced and the holes 55c exposed.

It will be seen that the reserve volume 7′ of an empty container 7 or bag (FIG. 5) will be replenished successively through the medium of the valve arrangement 41 and the flow of insufflation gas passing from the ventilator to the container 7 or bag, via the hose 44a and through the holes 55c.

Thus, the valve arrangement 41 can include a number of different valve functions, all of which are coordinated for actuation with the aid of the up-and-down movement of the piston 52 in the cylinder 51.

A number of valve functions can be activated in the upper position of the piston 52, in accordance with the FIG. 5 illustration.

In this regard, a first valve function “V1” lies in the upward movement of the piston 52, said piston exposing the holes 52a″ for a manual insufflation phase in the upper piston position.

A second valve function “V2” lies in movement of the piston 52, in which the channel 48 is exposed for air ventilation (53) and an overpressure is applied from the container 7 or bag.

A third valve function “V3” lies in the lower position of the piston, wherein, when the valve 55 at the top of the piston is open, insufflation gas can enter the container 7 or bag through the hose 44a.

A fourth valve function “V4” is independent of the positional setting of the piston 52 in the cylinder 51 and is function-mode-related as a second function-mode-related valve.

The valve function “V4” is of the functional mode “CMV” and is activated to a first state, a closed state, via a remotely controlled device, illustrated in FIG. 7 as a pneumatically activatable piston-cylinder-device, in which a piston 71 can be urged against a seating 74 by a spring 73, to effect a seal in the functional mode “CMV” and is moved from this sealing position in the functional mode “Spontaneous/Manual”.

Alternatively, a valve body 72 can be actuated in a similar way with the aid of an attraction or pulling magnet and a spring, and is activated to a second position, an open position according to FIG. 7, as in the functional mode “Spontaneous/Manual”.

FIG. 8 is intended to illustrate that a seal can be achieved between the piston 52 and the inner cylinder surface 51a, with the aid of oblique surface parts 52d and 51d.

It will be understood that the invention is not restricted to the aforedescribed and illustrated exemplifying embodiment thereof and that modifications can be made within the scope of the inventive concept as illustrated in the accompanying claims.

1. A lung ventilator adapted for use on a patient that is under the effect of intravenous anaesthesia, comprising a control unit, at least one gas delivery hose, and a patient-allocated or -related device, wherein one end part of the hose faces towards the control unit while the other end part of the hose faces towards the patient-allocated or -related device, wherein said control unit includes means for delivering to said patient an insufflation gas flow, and wherein an expiration valve is adapted to allow expiration gas to flow freely to the surroundings, and wherein said expiration valve can be controlled to take one of two states, characterized in that a pneumatic system, actuated by the control unit, is connected to a patient-allocated-pneumatic system via a valve arrangement; and in that a container, for containing a reserve volume, is coupled to said valve arrangement so as to provide a manually initiated insufflation phase, via said arrangement.

2. A ventilator according to claim 1, characterized in that the expiration valve can be controlled, via an overpressure in a hose, and is orientated close to or adjacent to the patient.

3. A ventilator according to claim 1, characterized in that the container or bag reserve volume is intended to generate, for a manual insufflation phase, a gas flow that can be actuated by applying pressure to the container temporarily and over a short time period; and in that the reserve volume, in said container or bag, is delivered to the hose via the valve arrangement during said pressurisation of the reserve volume.

4. A ventilator according to claim 1, characterized in that the container is connected to a T-coupling belonging to the insufflation hose.

5. A ventilator according to claim 1, characterized in that the control unit including the valve arrangement includes a piston, which allows the passage of insufflation gas to the patient.

6. A ventilator according to claim 1, characterized in that in one state of the valve arrangement said arrangement functions to allow gas to flow to the container or bag.

7. A ventilator according to claim 1, characterized in that the valve arrangement includes a number of valve functions.

8. A ventilator according to claim 1, characterized in that a valve allocated to the control unit is adapted to deliver a flow of insufflation gas to a hose and to said patient-allocated or -related device.

9. A ventilator according to claim 1, characterized in that a portion of said insufflation flow is adapted for delivery to said container or bag via a valve arrangement.

10. A ventilator according to claim 1, characterized in that an expiration valve has the form of a membrane.

11. An arrangement according to claim 10, characterized in that said membrane is cupped shaped.

12. An arrangement according to claim 1, characterized in that said valve arrangement has a first valve function, which is adapted to expose piston-associated holes (52″) for a manual insufflation phase.

13. An arrangement according to claim 1, characterized in that said valve arrangement has a second valve function, which is adapted to allow a channel (48) to be connected to an overpressure generated in the container or bag.

14. An arrangement according to claim 13, characterized in that the channel is opened for causing an air ventilating position.

15. An arrangement according to claim 1, characterized in that said valve arrangement includes a third valve function, which is related adjacent to a piston cavity part close to the piston top.

16. An arrangement according to claim 15, characterized in that the valve function includes a valve body, which, when in an upper position, can seal against holes that face towards the piston top and to expose said holes in a lower piston position.

17. An arrangement according to claim 15, characterized in that insufflation gas can pass into the container or bag, when the valve body is in a position in which the holes are exposed.

18. An arrangement according to claim 1, characterized in that the valve arrangement includes a cylinder and a piston that is movable therein, wherein the piston is adapted to take an upper position in response to a through-passing gas flow and to take a lower position in the absence of said gas flow.

19. An arrangement according to claim 1, characterized in that the valve arrangement includes a fourth valve function, which can be actuated to a first state in the function mode “CMV”, and actuated to a second state in the functional mode “Spontaneous/Manual”.

20. An arrangement according to claim 19, characterized in that the first state is a state in which the valve closes towards the container or bag.

21. An arrangement according to claim 1, characterized in that an obliquely configured seal acts between a piston and a cylinder in the valve arrangement.

22. A ventilator according to claim 9, characterized in that a chosen opening resistance of a one-way valve is adapted primarily to control the container-replenishing rate.

23. A ventilator according to claim 1, characterized in that a chosen continuous flow for a one-way valve and a well-balanced pneumatic resistance relationship in the pneumatic system are mutually adapted to limit the maximum pressurisation within the container.
